(19) United States
(12) Patent Application Publication
(10) Pub. No.: US 2016/0320417 A1
(43) Pub. Date: Nov. 3, 2016

(54) METHODS AND KITS FOR DETERMINING RISK FOR DEVELOPING ALZHEIMER'S DISEASE AND PREVENTION OR TREATMENT THEREOF

(71) Applicant: Loma Linda University, Loma Linda, CA (US)

(72) Inventors: Salvador Soriano, Irvine, CA (US); Michael Castello, Loma Linda, CA (US)

(21) Appl. No.: 15/143,234

(22) Filed: Apr. 29, 2016

Related U.S. Application Data
(62) Division of application No. 14/316,520, filed on Jun. 26, 2014.
(60) Provisional application No. 61/839,724, filed on Jun. 26, 2013.

(51) Int. Cl. G01N 33/92; A61K 31/40; A61K 31/455; A61K 31/366

(52) U.S. Cl. CPC G01N 33/92; A61K 31/366; A61K 31/40; A61K 31/455; G01N 2800/2821; G01N 2800/50

(57) ABSTRACT

Methods are disclosed for determining a patient's risk for developing Alzheimer's disease and preventing or treating Alzheimer's disease. Kits for assessing a patient's risk of developing Alzheimer's are also provided.

### CROSS-REFERENCE TO RELATED PATENT APPLICATIONS

This patent application is a divisional of U.S. patent application Ser. No. 14/316,520 filed on Jun. 26, 2014, which claims priority to U.S. Provisional Patent Application No. 61/839,724 filed on Jun. 26, 2013, entitled “METHODS AND KITS FOR DETERMINING RISK FOR DEVELOPING ALZHEIMER'S DISEASE AND PREVENTION OR TREATMENT THEREOF”, the disclosures of which are herein incorporated by reference in their entirety.

### BACKGROUND

1. Field of the Invention

The present disclosure relates to methods and kits for assessing a patient's risk for developing Alzheimer's disease, for preventing or delaying the onset of Alzheimer's disease, or for reducing the symptoms of Alzheimer's disease.

2. Description of the Related Art

Alzheimer's disease (“AD”) is an irreversible, progressive brain disease and is a form of dementia, the loss of cognitive functioning and behavioral abilities that affects daily life and activities. Alzheimer's disease affects 5.4 million Americans. Because of no known cure or effective treatment, the disease incurs an estimated annual cost of $183 billion. Affirmative diagnosis of the disease is only possible at autopsy. Previous studies suggested that the accumulation of the amyloid peptide was responsible for the onset of the disease, and consequently, research in the last 20 years has primarily focused on the prevention of amyloid peptide accumulation in the brain to ameliorate or halt the disease. These approaches have not been successful.

### SUMMARY

Methods and kits for determining a patient's risk of developing Alzheimer's disease (“AD”), prevention or delay of onset of AD, and reducing the symptoms of AD in a patient in need thereof are disclosed.

In some embodiments, a method for determining a patient's risk of developing Alzheimer's disease is provided. The method comprises obtaining a test sample from the patient; combining the test sample with an intracellular cholesterol staining reagent to form a labeled cholesterol complex; estimating a concentration of intracellular cholesterol in the test sample by quantifying the amount of labeled cholesterol complex in the test sample; and comparing the estimated concentration of intracellular cholesterol in the test sample with a concentration of intracellular cholesterol in a control sample, wherein a greater concentration of intracellular cholesterol in the test sample compared to the control sample indicates an increased risk of developing Alzheimer's disease.

In some embodiments, a method wherein the patient suffers from mild cognitive impairment. In some embodiments, a method wherein the intracellular cholesterol staining reagent is selected from filipin, BCθ and Amplex® red reagent. In some embodiments, a method wherein the staining reagent is filipin. In some embodiments, a method wherein flow cytometry is used to quantify the amount of filipin-labeled cholesterol complex in the test sample. In some embodiments, a method wherein the test sample comprises peripheral blood cells.

In some embodiments, a method for preventing or delaying the onset of Alzheimer's disease, or reducing the symptoms of Alzheimer's disease in a patient in need thereof is provided. The method comprises obtaining a test sample from the patient; determining a level of intracellular cholesterol in the test sample; and comparing the level of intracellular cholesterol in the test sample with a control level of intracellular cholesterol, identifying at least one cholesterol defect or distress of the patient; and administering at least one therapeutic agent which targets the at least one cholesterol defect or distress.

In some embodiments, a method wherein the patient suffers from mild cognitive impairment. In some embodiments, a method wherein a filipin test is used to determine the level of intracellular cholesterol. In some embodiments, a method wherein said at least one therapeutic agent is a cholesterol-lowering drug. In some embodiments, a method wherein the test sample comprises peripheral blood cells.

Further provided is a kit for assessing a patient's risk of developing Alzheimer's disease. The kit comprises a tissue collecting assembly; an intracellular cholesterol staining reagent; and a composite control slide. In some embodiments, the kit further comprising a tool for visualizing a cholesterol stain. In some embodiments, a kit wherein the tissue collecting assembly comprises a venipuncture assembly to collect peripheral blood cells as a test sample.

### BRIEF DESCRIPTION OF THE DRAWINGS

FIG. 1 shows how cholesterol distress, rather than Aβ accumulation, may be the more likely candidate for a bona fide pathogenic trigger in most cases of late-onset AD.

FIG. 2A-B depicts representative images of a filipin test for a patient with severe Niemann-Pick type C (“NPC”) disease (“NPC 1 year” or “NPC01”) and less severe Niemann-Pick type C disease (“NPC 21 years” or “NPC21”).

FIG. 2C-D also depicts representative images of a filipin test for a healthy patient age-and-sex matched to patient NPC1 year (“Control 1 year” or “CTL01”) and age-and-sex matched to patient NPC 21 years (“Control 23 years” or “CTL23”).

FIG. 2E is a graph showing the filipin-positive percentages for patients NPC01, CTL01, NPC21, and CTL23.

FIG. 3A-B depicts representative figures of a filipin test of a 73-year-old patient with Alzheimer's disease (“AD 73 years” or “AD73”) and a healthy patient age-and-sex matched to patient AD73 (“Control 73 years” or “CTL73”)).

FIG. 3C is a graph showing the filipin-positive percentages for patients CTL73 and AD73.

FIG. 4 shows the filipin score of fibroblasts taken from three patients affected with Alzheimer's disease (“08245(AD),” “08243(AD),” “07375(AD)”) and one age-matched cognitively healthy patient (“0919(CTL)”).

FIG. 5A depicts a fluorescence-activated cell sorting (“FACS”) analysis of fibroblasts taken from a patient affected with Alzheimer's disease (“AG08245”).

FIG. 5B depicts a fluorescence-activated cell sorting (“FACS”) analysis of fibroblasts taken from one age-matched cognitively healthy patient (“AG0919”).

FIG. 5C depicts a fluorescence-activated cell sorting (“FACS”) analysis of fibroblasts taken from a patient affected with Alzheimer's disease (“AG08243”).

FIG. 5D depicts a fluorescence-activated cell sorting (“FACS”) analysis of fibroblasts taken from a patient affected with Alzheimer's disease (“AG07375”).

FIG. 6 depicts an exemplary graph of the prophetic data displayed in Table 2.

FIG. 7 shows that a dose-dependent effect of U18666A can be demonstrated on filipin staining in peripheral blood cells.

FIGS. 8A-B show the Filipin-positive B-Lymphocytes in control and AD patients and the average mean intensity of filipin fluorescence in control and AD patients.

### DETAILED DESCRIPTION OF THE PREFERRED EMBODIMENT

### Disease and Anatomy

Alzheimer's disease (AD) is the most common form of dementia in the United States, representing around eighty percent of all cases. As the country's sixth leading cause of death, AD currently affects 5.4 million Americans, or one in eight people 65 or older (Alzheimer's Association, 2011). With no cure or effective treatment, AD incurs an estimated annual cost of $183 billion (Alzheimer's Association, 2011). As the US population continues to age, the number of individuals with AD is projected to increase to 7.7 million over the next twenty years (Alzheimer's Association, 2011).

Affirmative diagnosis of the disease is only possible at autopsy, when specific pathological hallmarks can be visualized in the brain: extracellular senile plaques (SPs), comprised of the amyloid peptide Aβ, and neurofibrillary tangles (NFTs), made from abnormally phosphorylated tau protein. Aβ is a 38 to 43 amino acid peptide formed through the sequential cleavage of the amyloid precursor protein (APP) by β- and γ-secretases. The 40 (Aβ₄₀) and 42 (Aβ₄₂) amino acid forms are the most abundant, the latter having the greater tendency to aggregate.

NFTs are formed when the tau protein is abnormally phosphorylated and aggregates into paired helical fragments. Tau is a cytoskeletal protein with a key role in axonal transport; formation of NFTs disrupts this transport and structurally disables neurons, leading to cell loss, cognitive decline, and patient death.

### The Amyloid Cascade Hypothesis

The identification of SPs and NFTs in AD led to the proposal of the Amyloid Cascade Hypothesis. This hypothesis states that AD initiates when Aβ accumulates and aggregates to form SPs, which in turn induce the formation of NFTs and cell death, thereby causing the pathological presentation and dementia. For the past several decades, researchers have been guided by the primary prediction of this hypothesis: preventing Aβ aggregation will ameliorate or halt progression of the disease.

Supporting this hypothesis, trisomy 21 (Down syndrome) usually results in three copies of the APP gene, and consistently coincides with AD-like pathology. Mechanistic studies have also shown that Aβ accumulation can precede, and lead to, tau phosphorylation and aggregation into NFTs. Additionally, rare forms of familial early-onset AD are linked to specific mutations in APP or to enzymes that promote Aβ aggregation. Proponents of the amyloid cascade hypothesis have assumed that late-onset AD is mechanistically identical to early-onset, consistently placing Aβ as the origin of pathogenesis in all cases.

### Shortcomings of the Amyloid Cascade Hypothesis

Conversely, there is an increasing amount of data contradicting the notion that Aβ is the key pathogenic trigger in late-onset AD, which represents the vast majority of cases. Notably, there is no significant correlation between Aβ accumulation and cognitive deterioration in either humans or in mouse models, and the appearance of SPs has been reported in the brains of approximately thirty percent of individuals with no dementia. Such findings have prompted some to reasonably advocate restructuring AD is to focus on symptomatic changes rather than the neuropathological identification of SPs and NFTs.

Despite the waning support of the amyloid cascade hypothesis at the mechanistic level, there has been optimism that pharmacologically correcting Aβ abnormalities might nevertheless help cure or slow progression of the disease. The most promising Aβ-modifying drugs that have reached clinical trial stages have targeted Aβ production, inhibition of the enzymes involved in its generation (β- or γ-secretases), or driving γ-secretase activity toward the production of shorter, less aggregation-prone forms of Aβ in lieu of Aβ₄₂. Other strategies have included the inhibition of Aβ aggregation, the induction of an immune response against it, or enhancing mechanisms of Aβ removal.

Discouragingly, the clinical trials of these drugs have produced lackluster outcomes. Recent compounds targeting γ-secretase, such as tarenflurbil, even when successfully reducing Aβ levels, have shown no benefit to cognitive function. While antibodies targeted against aggregating Aβ have managed to decrease Aβ plaques, there has been no significant change in survival or cognition, NFTs have remained in plaque-free areas, and vascular deposition of amyloid, known as cerebral amyloid angiopathy (CAA), has actually increased. Other immunotherapy treatments have caused vasogenic edema while showing no evidence of therapeutic benefit. Drugs like scyllo-inositol and homotaurine, aimed at preventing Aβ aggregates from forming into SPs, have failed to mitigate clinical AD progression.

The amyloid cascade hypothesis also lacks at the conceptual level, not having a theoretical framework in which the physiological generation of Aβ is well understood, and the mechanisms leading to its accumulation are placed in that context. This shortcoming stems from our poor understanding of APP biology; despite the wealth of data on the cellular and molecular mechanisms of APP trafficking and processing, the function of APP is in the brain is not known. Until it is known, it is difficult to understand why Aβ is generated in the healthy brain, let alone place it within the pathogenic cascade of AD. Nevertheless, current evidence argues against a central pathogenic role for Aβ in late-onset AD. For example, neurodegeneration begins with an initiating injury, subsequently leading to microglial dysregulation and inflammatory responses, and, eventually, resulting in a so-called “change-of-state” in cells that primes them for neurodegeneration. While this model does not provide specifics on the nature of an initiating injury that would be relevant to the majority of AD cases, it represents a comprehensive interpretation of available data; it identifies Aβ accumulation as a potential part of the process of microglial dysregulation and inflammation that characterizes the AD brain, while rejecting it as neither central to, nor a requirement for, the initiation and progression of the disease.

### The Candidates for Pathogenic Triggers Beyond the Amyloid Cascade Hypothesis

The amyloid cascade hypothesis defines AD as a series of pathological hallmarks that are causally linked, both in familial (early-onset) and sporadic (late-onset) cases. It assumes that erasing those hallmarks will restore cognitive function. Current evidence does not support this concept for sporadic cases; rather, the data suggest that Aβ is either too peripheral or too close to the final stages of the disease to be considered the primary initiator of pathogenesis, making it of little therapeutic use. Therefore, likely candidates for bona fide pathogenic triggers in late-onset AD can be identified.

### Cholesterol Dysregulation: A Common Pathogenic Trigger in Late-Onset Alzheimer's Disease

It is well documented that cholesterol is a key regulator of cellular processes in the brain, including the formation of myelin sheaths, synaptic function, membrane fluidity and neurosteroid biosynthesis; cholesterol homeostasis has also been linked to learning and memory function in both humans and animals.

It is noteworthy that, while physiological cholesterol synthesis rates and levels in the healthy brain decline more than 40% with age, reflecting the different needs and roles for brain cholesterol at different stages of life, this phenotype is reversed in late-onset AD. Furthermore, there is a strong correlation between levels of brain cholesterol and disease severity in the AD brain.

That brain cholesterol levels may evolve differently in healthy elderly individuals and late-onset AD patients can be suggestive of a causative role for cholesterol in the pathogenesis of the disease. This causative role can be more clearly illustrated by several other lines of evidence. At the cellular level, cholesterol dysregulation can both precede and affect SPs and NFTs, the two key pathological hallmarks of AD: The amyloidogenic processing of APP occurs in cholesterol-rich lipid rafts, and cholesterol accumulation can enlarge lipid raft size, thereby increasing Aβ generation. Supporting this concept, rabbits and APP transgenic mice fed a cholesterol-enriched diet showed increases in Aβ Furthermore, anomalies in cholesterol levels and distribution are able to influence the phosphorylation state of tau and create NFTs independently of Aβ.

There is also a well-established link between AD and apolipoprotein E (ApoE), a protein that, in the brain, is involved in transporting cholesterol and other lipids. Individuals homozygous for the ε4 allele of the APOE gene, which produces a protein product that is a less efficient transporter than its counterparts, are as much as 18-fold more likely to develop AD. More recent genetic evidence in support of cholesterol dysregulation as causative of late-onset AD has been provided by genome-wide association studies. Remarkably, in addition to APOE, four other loci are associated with cholesterol metabolism and/or transport: CLU, ABCA7, LDLR, SORL1. A second associated pathway is that of endocytosis, also linked to cholesterol biology. In that respect, PICALM is of particular interest, as it has been shown to confer risk predominantly in the presence of APOE ε4 positive subjects.

Other neurodegenerative conditions can also be informative with regards to a potential causative role for cholesterol in brain pathogenesis. Of particular interest is the case of Niemann-Pick type C disease (“NPC”). In NPC, mutations in one of two genes, NPC1 and NPC2, results in cholesterol and other lipids accumulating in late endosomes and lysosomes. In common with AD, APP is disproportionately metabolized to Aβ in NPC, even forming AD-like plaques in patients carrying the ApoE ε4 allele; tau has been observed both in early stages of hyper-phosphorylation and as NFTs that are indistinguishable from those appearing in AD brains. NFTs are also more readily detectable in cell populations most affected by cholesterol accumulation, often forming without any prior deposition of Aβ into plaques, indicative of a direct causative link between cholesterol dysregulation and NFT formation.

In addition, increased expression of NPC1 has been reported in cortex and hippocampus from AD patients, and some NPC1 polymorphisms have been linked to increased risk of developing AD. Finally, loss of one copy of the Npc1 gene in AD mice harboring PS1 and APP mutations leads to accelerated Aβ₄₂ accumulation, compared to control mice with two copies of Npc1.

In summary, cholesterol can be causally linked to neurodegeneration in late-onset AD at several levels. Besides its direct impact on amyloidogenesis and tau aggregation, a significant number of genes involved in cholesterol homeostasis are associated with AD, and other neurodegenerative conditions such as NPC provide formal proof that cholesterol dysregulation precedes, and leads to, Aβ and tau abnormalities.

### Dysregulation of APP can be a Key Contributor to Cholesterol-Driven Pathogenesis in Late-Onset AD

The biological function of APP in the brain, in particular with regards to the physiological generation of Aβ and the mechanisms leading to its accumulation, are not well understood; this shortcoming weakens the theoretical persuasiveness of the amyloid cascade hypothesis. At the same time, it hinders the progress of alternative views that better explain the role of APP and Aβ in health and disease.

The evidence available indicates a potential role for APP as a regulator of cholesterol homeostasis in the brain through mechanisms including, but not limited to, Aβ generation. The evidence in support of APP as a cholesterol regulator in the brain is multifold. In a mouse model of NPC that faithfully recreates the cholesterol accumulation and disease phenotype observed in human patients, loss of APP leads to an exacerbation of the cholesterol abnormalities seen in NPC brains, which in turn leads to accelerated progression of the disease. Furthermore, a comparison of gene expression profiles in pre-symptomatic cerebella of control mice to NPC mice with and without APP reveals that the latter may be necessary for appropriate cholesterol synthesis in the post-mevalonate pathway; cholesterol transport; myelination, and cholesterol regulation through modification of histone deacetylase.

The cleavage products of APP affect cholesterol synthesis and transport directly: Aβ acts to reduce the size of lipid rafts and decreases cholesterol synthesis by inhibiting the rate-limiting enzyme, HMG CoA reductase (HMGR). The remaining intracellular domain (AICD) acts as a transcription factor to suppress production of the cholesterol transporter LRP1. Furthermore, Aβ production is increased in response to 27-hydroxycholesterol, an oxidized, neurotoxic form of cholesterol that is increased in AD brains.

Finally, APP contains a cholesterol-sensing domain shown to be functional in vitro, thereby providing a potential molecular mechanism by which APP could be involved in regulation of brain cholesterol through HMGR, LRP1, and other cholesterol-associated pathways.

### The Cholesterol Hypothesis of Neurodegeneration

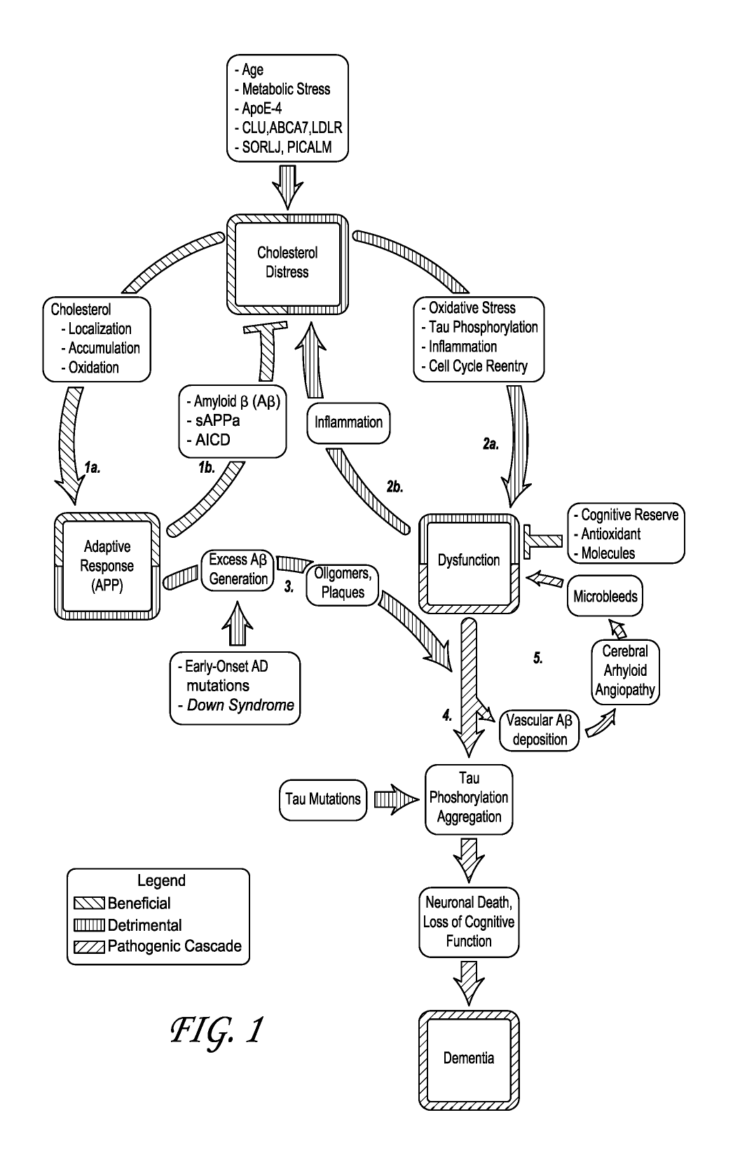

The model, outlined in FIG. 1, shows that sustained cholesterol distress, rather than Aβ accumulation, can be the more likely candidate for bona fide pathogenic trigger in most cases of late-onset AD. The model is based on the fact that cholesterol dysregulation can be causally linked to pathogenesis in the AD brain and that APP may function as a regulator of cholesterol homeostasis leads us to propose a novel model of neurodegeneration.

FIG. 1 shows how cholesterol distress, rather than Aβ accumulation, may be the more likely candidate for a bona fide pathogenic trigger in most cases of late-onset AD. Cholesterol distress may be defined as some combination of abnormal amounts of cholesterol or its precursors and metabolites, aberrant subcellular localization, or oxidation state (arrow 1a), and is primarily caused by multiple factors, including age, metabolic stress, and gene polymorphisms. In turn, cholesterol distress prompts activation of an APP-driven adaptive response, through a mechanism involving, in part, the generation of Aβ (arrow 1b). If the adaptive response from APP is insufficient, cholesterol distress can result in cell dysfunction (arrow 2a). Cell dysfunction can be defined by the presence of major cellular damage as a consequence of axonal transport disruption, tau phosphorylation and aggregation, inflammatory response, reactive oxygen species, and neuronal commitment to aberrant entry into the cell cycle. Cell dysfunction can in turn affect cholesterol regulation via a mechanism mostly driven by inflammation (arrow 2b). Note that the pathogenic cascade only initiates after cell dysfunction, leading to synaptic collapse, loss of dendritic mass, and neuronal destruction that results in dementia and death (pathway defined by arrow 4). Aβ oligomers and plaques form when there is an excessive defensive response from APP to chronic cholesterol distress. In a brain where cell dysfunction is already ongoing (i.e. within the pathogenic cascade leading to dementia shown by arrow 4), excess Aβ affects already-vulnerable neurons, further inducing inflammatory responses, extending oxidative damage, and contributing to tau hyperphosphorylation and aggregation. Vascular Aβ deposition would be prominent at this stage, contributing to cerebral amyloid angiopathy and microbleeds, exacerbating the pathogenic cascade as noted by loop 5. Note that, where plaques are present in cognitively functional individuals, an excessive adaptive response from APP has generated a large amount of Aβ, while successfully defending against cholesterol distress. In this case, cell dysfunction is absent and the formation of Aβ oligomers and plaques does not have an obvious impact on neuronal function (not shown in figure). In early-onset AD and Down syndrome, Aβ is constitutively overproduced throughout life (or the Aβ₄₂/Aβ₄₀ ratio is significantly higher), to levels that are sufficient to cause dysfunction.

Cholesterol distress includes but is not limited to some combination of abnormal amounts of cholesterol or its precursors and metabolites, aberrant subcellular localization, or oxidation state, all of which are involved in the pathogenesis of neurodegeneration as shown in FIG. 1, arrow 1a. This cholesterol distress can be caused by multiple factors, including aging, metabolic stress and gene polymorphisms as illustrated by the top of FIG. 1. In turn, cholesterol distress prompts activation of an APP-driven adaptive response, through a mechanism involving, in part, the generation of Aβ as illustrated in FIG. 1 by arrow 1b. The view of Aβ as a protective molecule in the brain has been proposed before. The Aβ accumulation is a byproduct of upstream pathogenic events and initiates as a protective response to neuronal insult. Such a notion is also consistent with data showing that Aβ is neuroprotective at physiological concentrations and that Aβ can be generated in response to acute damage caused by ischemia and head trauma. Nevertheless, the nature of the stress stimuli that would initiate an Aβ response in late-onset AD remains unclear. It has been proposed that the underlying stress is of an energetic nature through mechanisms involving oxidative stress. APP is a key part of the protective response against cholesterol distress via Aβ regulation of HMGR and LRP1, and potentially other mechanisms involving cholesterol synthesis, transport or histone deacetylase regulation.

If the adaptive response from APP is insufficient, cholesterol distress can result in cell dysfunction. Cell dysfunction can include but is not limited to the presence of major cellular damage as a consequence of axonal transport disruption, tau phosphorylation and aggregation, inflammatory response, reactive oxygen species, and neuronal commitment to aberrant entry into the cell cycle—all of which have been shown to occur as a consequence of cholesterol distress. This is illustrated by FIG. 1, line 2a. The link between cholesterol and the neuronal cell cycle, an early event in AD known to be a cause, rather than a consequence, of neurodegeneration can be of interest. Disruption of cell-cycle reentry genes has been reported in young adults homozygous for the ε4 allele of the APOE gene. A mechanism for cholesterol-induced cell cycle reentry in AD involves miR-33, a microRNA encoded within SREBP-2 that is involved in the regulation of cholesterol synthesis and uptake, as well as the expression of cell cycle genes—including cyclin D1. Specifically, downregulation of miR-33 leads to increased cyclin D1 levels, and could conceivably result in neuronal reentry into the cell cycle. SREBP-2 is downregulated in AD, and since miR-33 levels mirror those of SREBP-2, the resulting increase in cyclin D1 would contribute to an aberrant entry into the cell cycle.

Cell dysfunction can in turn affect cholesterol regulation via a mechanism mostly driven by inflammation as shown in FIG. 1, line 2b. Note also that the pathogenic cascade only initiates after cell dysfunction, leading to synaptic collapse, loss of dendritic mass, and neuronal destruction that results in dementia and death as illustrated by FIG. 1 and the pathway defined by line 4.

The model of FIG. 1 accounts for Aβ oligomers and plaques forming when there is an excessive defensive response from APP to chronic cholesterol distress shown by line 3. In a brain where cell dysfunction is already ongoing (i.e. within the pathogenic cascade leading to dementia; line 4 in FIG. 1), excess Aβ affects already-vulnerable neurons, further inducing inflammatory responses, extending oxidative damage, and contributing to tau hyperphosphorylation and aggregation. Vascular Aβ deposition would be prominent at this stage, contributing to cerebral amyloid angiopathy and microbleeds, exacerbating the pathogenic cascade as demonstrated in FIG. 1 by pathway 5.

Where plaques are present in cognitively functional individuals, an excessive defensive response from APP has generated a large amount of Aβ, while successfully defending against cholesterol distress. In this case, cell dysfunction is absent and the formation of Aβ oligomers and plaques does not have an obvious impact on neuronal function (not shown in FIG. 1).

Early-onset AD and Down syndrome are fundamentally different from late-onset AD. In the former two cases, Aβ is constitutively overproduced throughout life (or the Aβ₄₂/Aβ₄₀ ratio is significantly higher), in the absence of cholesterol distress. In such instances, the protective role of APP metabolites in the brain is not available, and the sustained overexpression of Aβ accelerates the rate at which cell dysfunction occurs, through a combination of a weaker adaptive response (represented by line 1b in FIG. 1) and an accelerated rate at which cell dysfunction occurs (represented by line 2a in FIG. 1). Neurodegeneration is further exacerbated by the impact of a higher Aβ load (line 3 in FIG. 1) on the pathogenic cascade (represented by line 4 in FIG. 1).

The opposite situation occurs in the case of the rare mutation within the App gene, A673T. This mutation is less common in AD patients than in non-demented individuals, and elderly carriers perform better in cognitive tests than do non-carriers. Although the impact of this mutation on the APP and cholesterol metabolite profiles in the brain is unknown, amyloidogenesis from A673T APP is significantly reduced in cultured cells, whereas α-secretase cleavage is modestly decreased. In mutant carriers, Aβ is underproduced throughout life, while preserving the overall function of APP within the adaptive response to cholesterol distress. As a consequence, there is no Aβ excess secondary to a sustained adaptive response (illustrated in arrow 3, FIG. 1), and the potential contribution of Aβ to the pathogenic cascade (arrow 4 in FIG. 1) and CAA (loop 5 in FIG. 1) is drastically reduced, accounting for the slower rate of neurodegeneration and the delay in AD age of onset, as reported. Note also that the A673T mutation creating a stronger adaptive response is also consistent with the superior performance in cognitive tests of patients with the mutation, who retain cognitive ability for a longer period of time even when they do develop AD. Broader applications of the cholesterol distress or failure of cholesterol regulation

The status of cholesterol as a critical functional element in the brain can allow for cholesterol to also be used as a risk or causative factor for neurodegenerative conditions other than AD. In Parkinson's disease (PD) and the related Lewy body dementia, for example, there is a progressive degeneration of dopaminergic neurons, accompanied by intra-neuronal deposition of α-synuclein (α-syn). Increases in cholesterol metabolites have been found in these cells, which, in vitro, promote α-syn aggregation. As is the case with APP, α-syn is able to associate with the cholesterol-containing lipid rafts, and is upregulated in the brain in response to 27-hydroxycholesterol, suggesting that it may be part of an adaptive response to cholesterol distress analogous to that occurring in AD.

Other diseases that have been proposed to be causally linked to cholesterol distress are Huntington's disease, schizophrenia, and autism spectrum disorder (ASD). Additionally, ASD has a high coincidence with Smith-Lemili-Opitz syndrome, which is a direct consequence of a defect in cholesterol synthesis.

Further, as stated above, cholesterol can be involved in a variety of neurodegenerative conditions. ApoE is one of the critical genetic risk factors for AD. ApoE also appears as a risk or exacerbating factor for Parkinson's disease, cerebral amyloid angiopathy, tauopathies and other dementias, and even multiple sclerosis. Therefore, cholesterol can be used as a risk or causative factor for detecting and/or preventing Parkinson's disease, cerebral amyloid angiopathy, tauopathies and other dementias, multiple sclerosis, and other neurodegenerative conditions known in the art. There appears to be many instances of neurodegeneration that initiate or interface with a failure of cholesterol regulation. Molecules such as α-syn, huntintin, and others known in the art and/or discovered in the future would fill the role of APP and Aβ as part of a defensive response analogous to that proposed for AD. An excessive response to correct cholesterol imbalances would result in protein aggregation, with phenotypic differences determined by the type and nature of cholesterol stress and the defense response available in susceptible neuron populations.

Cholesterol distress can be a common early pathogenic factor in late-onset AD and a better target for intervention than Aβ From a therapeutic standpoint, due to the diversity of pathways through which cholesterol distress can occur, a single treatment may not prove beneficial to all AD sufferers. For example, while treatment with cholesterol-lowering statins may show a benefit in individuals with high cholesterol levels, these drugs may not work for patients for whom cholesterol distress is not specifically linked to increased cholesterol levels. As with cancer treatments, the research and pharmaceutical communities can utilize and develop cost-effective methods to test for a spectrum of cholesterol abnormalities, including levels, trafficking, oxidation, and synthesis that will form the basis of patient-specific medical care. The devices, methods, and/or treatments described herein can be used to test for a spectrum of cholesterol abnormalities to develop patient-specific medical care for a variety of neurodegenerative conditions, including, for example, AD. Devices, Methods, and Kits for Detection and Prevention of Alzheimer's disease and/or other diseases

According to the amyloid cascade hypothesis, accumulation of the amyloid peptide Aβ, derived by proteolytic processing from the amyloid precursor protein (APP), is the key pathogenic trigger in Alzheimer's disease (AD). This view has led researchers for more than two decades and continues to be the most influential model of neurodegeneration. Nevertheless, close scrutiny of the current evidence does not support a central pathogenic role for Aβ in late-onset AD. Furthermore, the amyloid cascade hypothesis lacks a theoretical foundation from which the physiological generation of Aβ can be understood, and therapeutic approaches based on its premises have failed.

An alternative model of neurodegeneration has been presented, in which sustained cholesterol-associated neuronal distress may be the most likely pathogenic trigger in late-onset AD, directly causing oxidative stress, inflammation and tau hyperphosphorylation. Aβ generation can be part of an APP-driven adaptive response to the initial cholesterol distress, and its accumulation can be neither central to, nor a requirement for, the initiation of the disease. The cholesterol model described herein provides a theoretical framework that allows APP to act as a regulator of cholesterol homeostasis, accounts for the generation of Aβ in both healthy and demented brains, and provides suitable targets for therapeutic intervention.

Because it was previously believed that AD began as a consequence of the accumulation of amyloid peptide in the brain, research has primarily been aimed at reducing amyloid peptide accumulation in the brain to ameliorate or halt the disease. However, such approaches have not been successful.

Mild Cognitive Impairment (“MCI”) is a disorder that causes cognitive changes serious enough to be noticed by the individual experiencing them but not severe enough to hinder their daily life or capability of functioning in society. MCI may be classified into two categories based on the thinking skills affected: “amnestic MCI” (where memory is impacted) and “nonamnestic MCI” (where thinking skills other than memory are impacted). A person with MCI generally may be at an increased risk of developing Alzheimer's disease. Every year, a small percentage of MCI patients develop Alzheimer's disease while others remain healthy. The risk of developing Alzheimer's disease in a person with MCI is currently unknown. Thus, a method of predicting a patient's risk of developing Alzheimer's, particularly that of a person suffering from MCI, is desirable.

Because cholesterol distress or cholesterol defects may be a likely cause of Alzheimer's disease, detection of such cholesterol defects and/or distress at an early stage of the disease or before the onset of the disease would allow for a prediction of a patient's risk for developing the disease, for preventing or delaying the onset of the disease, or reducing the symptoms of the disease.

### Methods

In some embodiments, the methods involve methods for assessing a patient's risk for developing Alzheimer's disease, for assessing a patient's risk of progressing from Mild Cognitive Impairment (MCI) to Alzheimer's disease, for preventing or delaying the onset of Alzheimer's disease, and/or for reducing the symptoms of Alzheimer's disease.

In some embodiments, a method for determining a patient's risk of developing Alzheimer's disease may comprise obtaining a test sample from the patient; determining a level of intracellular cholesterol in the test sample; and comparing the level of intracellular cholesterol in the test sample with a control level of intracellular cholesterol, wherein a greater level of intracellular cholesterol in the test sample compared to the control level indicates an increased risk of developing Alzheimer's disease.

In some embodiments, the level of intracellular cholesterol may comprise a level of intracellular cholesterol distress or a level of intracellular cholesterol defect. In some embodiments, the control level of intracellular cholesterol may comprise a control level of intracellular cholesterol distress or a control level of intracellular cholesterol defect.

In some embodiments, the patient may not be suffering from a disease. In other embodiments, the patient may suffer from, or may be prone to develop, MCI. In some embodiments, the patient may suffer from, or may be prone to develop, cholesterol distress and/or defect(s). The cholesterol defect, in some variations, may be a defect other than changes in the levels of intracellular cholesterol.

In some embodiments, obtaining a test sample from the patient may include collecting a sample of biological material, such as cells, DNA, and the like. Such biological material may be obtained from tissue or bodily fluid, including but not limited to blood, urine, saliva, and the like. The samples may be collected by methods known to those of skill in the art.

In some variations, determining a level of intracellular cholesterol in the test sample may include subjecting the test sample from a patient to a test to detect a level of intracellular cholesterol level. The test may be any known methods, such as assays, used to detect intracellular cholesterol levels and/or detect intracellular cholesterol levels as markers for the presence of a disease. In one embodiment, the test may involve combining the test sample (e.g., a patient's cell) with an intracellular cholesterol staining reagent to form a labeled cholesterol complex. Examples of intracellular cholesterol staining reagents include filipin, BCθ and Amplex® red reagent. Of course any staining reagent may be used. The concentration of intracellular cholesterol in the test sample may be estimated by quantifying the amount of labeled cholesterol complex in the test sample. In order to assess the risk of developing MCI and/or AD, the estimated intracellular cholesterol in the test sample may be compared with a parallel estimate of intracellular cholesterol in a control sample, e.g., from a healthy individual. The control sample may be a parallel sample, in which the amount of labeled cholesterol complex in the test sample and the control sample is analyzed together. In alternative embodiments, the control sample may be analyzed before or after analysis of the test sample. In still other embodiments, the control sample may be a reference standard derived from analysis of more than one healthy individuals, such that only the test sample may need to be stained and quantitated. In which case, risk is evaluated by comparing the quantitated level of labeled intracellular cholesterol with a reference value. In one embodiment, a greater concentration of intracellular cholesterol in the test sample compared to the control sample (or reference) indicates an increased risk of developing MCI and/or AD.

One non-limiting example of such tests is the administration and use of a reagent that binds to cholesterol to indicate the presence and/or amount of and/or chemical modifications to cholesterol. The presence and/or amount of and/or chemical modifications to cholesterol may be analyzed by known methods, including but not limited to flow cytometry or fluorescence microscopy.

In such embodiments, the test may be a filipin test. A filipin test uses filipin, an antibiotic polyene, as a histochemical marker for cholesterol. Filipin may be used to detect cholesterol via freeze fracture electron microscopy, fluorescence studies, flow cytometry, and the like. As a non-limiting example, a filipin test may be performed as follows: cells may be rinsed in phosphate buffered solution (“PBS”) and subsequently fixed with 4% paraformaldehyde for about 1 hour at room temperature. Cells may be rinsed with PBS again and incubated with a solution to quench the paraformaldehyde, such as 1.5 mg glycine/1 mL PBS solution, for 10 minutes at room temperature. The cells may then be stained with a solution of filipin (0.5 mg filipin/ml in a solution of PBS in 10% bovine serum albumin (BSA)) for 2 hours at room temperature. The cells may then be rinsed with PBS. The rinsed cells may then be analyzed for the presence of cholesterol via flow cytometry, fluorescence microsopy, and the like.

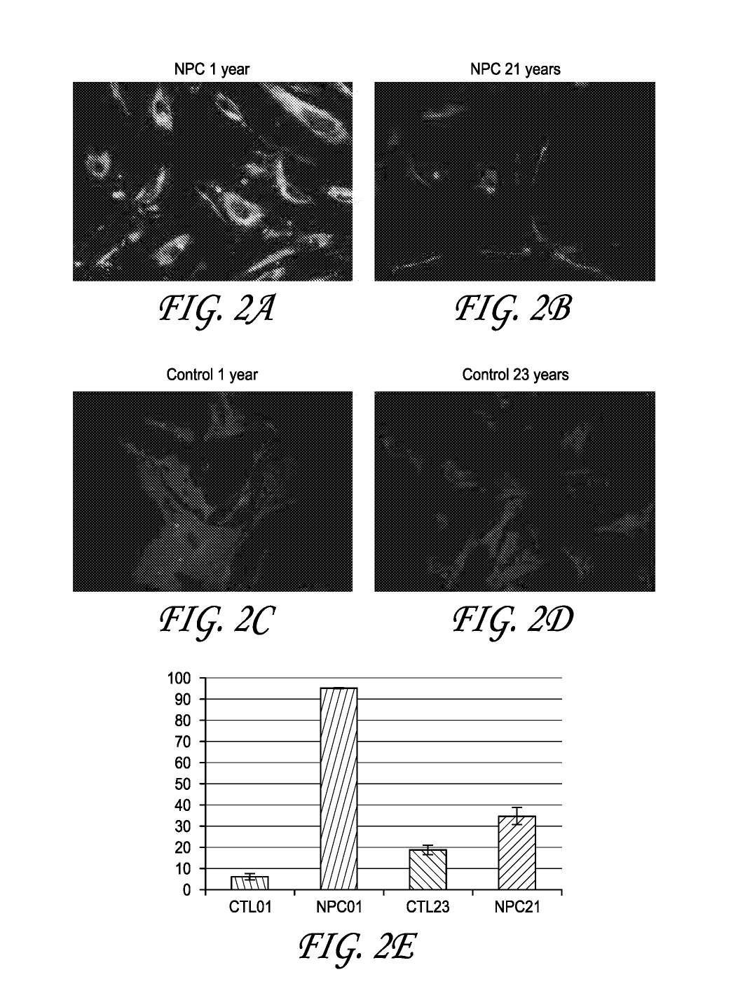

FIGS. 2A-B depicts representative images of a filipin test for a patient with severe Niemann-Pick type C (“NPC”) disease (“NPC 1 year” or “NPC01”) as shown in FIG. 2A and less severe Niemann-Pick type C disease (“NPC 21 years” or “NPC21”) illustrated in FIG. 2B. NPC is neurodegenerative condition and is characterized by cholesterol defects; the filipin test can be used as a diagnostic tool in children suffering from NPC. For comparison, FIGS. 2C-D also depicts representative images of a filipin test for a healthy patient age-and-sex matched to patient NPC1 year (“Control 1 year” or “CTL01”) as shown in FIG. 2C and age-and-sex matched to patient NPC 21 years (“Control 23 years” or “CTL23”) as shown in FIG. 2D. FIG. 2E is a graph showing the filipin-positive percentages for patients NPC01, CTL01, NPC21, and CTL23 (p<0.00001). Because NPC is a disease involving cholesterol distress, filipin staining in NPC cases can be used as a positive control in the measurement of filipin staining in AD cases.

As can be seen in FIGS. 2A-D, the white area identifies one single cell harboring cholesterol defects. In both control cases, there is a complete absence of white areas, thereby demonstrating that such individuals do not have a disease. For a patient suffering from a more severe Niemann-Pick type C disease, the filipin score was much higher (˜95%) than that of a patient suffering from a less severe Niemann Pick type C disease (˜35%). Thus, the severity of the disease can correlate with a patient's filipin score.

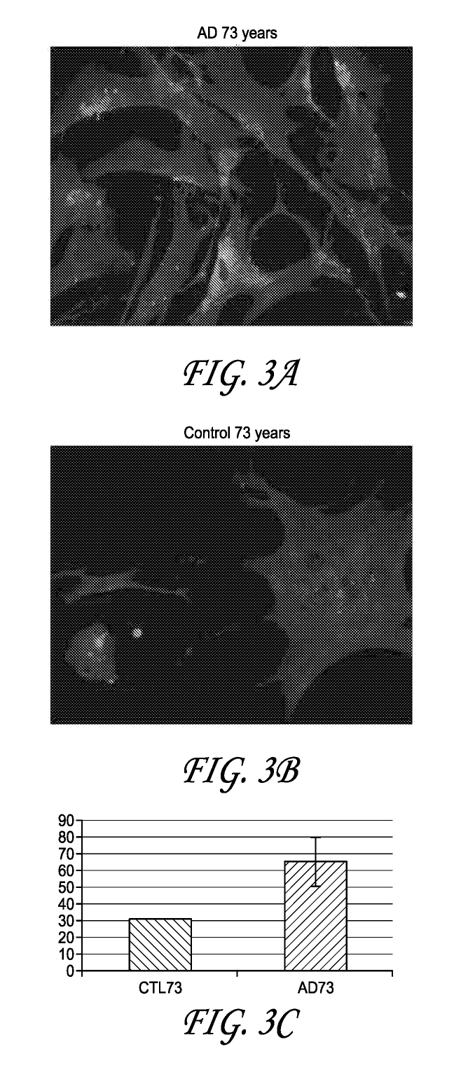

FIGS. 3A-B depicts representative figures of a filipin test of a 73-year-old patient with Alzheimer's disease (“AD 73 years” or “AD73”) as shown in FIG. 3A and a healthy patient age-and-sex matched to patient AD73 (“Control 73 years” or “CTL73”) as shown in FIG. 3B. FIG. 3C is a graph showing the filipin-positive percentages for patients CTL73 and AD73. The severity of the disease presentation as measured by random chromosomal loss and/or gain correlates with the degree of filipin staining. As can be shown from FIGS. 3A and 3B, the white areas are much more prominent in AD73 as compared to CTL73, and the filipin score for AD73 (˜65%) is also greater than that of CTL73 (˜31%). Thus, a filipin score can be used as a positive indicator for AD.

In other embodiments, other cholesterol binding reagents may be used. As another non-limiting example, BCθ may be used as an alternative to filipin using the methods described in Reid et al., Journal of Lipid Research 45 (2004) 582-591, herein incorporated by reference in its entirety.

In some variations, various reagents that may form fluorescent complexes with other molecules besides cholesterol as indicators of the presence of intracellular cholesterol defects may be used. As a non-limiting example, Amplex® red reagent (10-acetyl-3,7-dihydroxyphenoxazine) is a highly sensitive and stable probe for H₂O₂; H₂O₂ is generated when cholesterol is oxidized by cholesterol oxidase (the cholesterol being formed when cholesteryl esters are hydrolyzed by cholesterol esterase). Amplex® red reagent forms a highly fluorescent resorufin upon reaction with H₂O₂.

The tests may be any assay kit used for the detection of cholesterol. Such assay kits may include, for example, Amplex® Red cholesterol assay kits or cholesterol cell-based detection assay kits that include filipin (e.g. from Caymen Chemical Company or Abnova Corporation).

In some embodiments, comparing the level of intracellular cholesterol in the test sample with a control level of intracellular cholesterol, wherein a greater level of intracellular cholesterol in the test sample compared to the control indicates an increased risk of developing Alzheimer's disease, may include comparing the patient's measured level of intracellular cholesterol based on the results obtained from subjecting the patient's test sample to a control level of intracellular cholesterol.

In some embodiments, the control level of intracellular cholesterol may be empirically derived. In such embodiments, a test to detect intracellular cholesterol levels may be carried out on patient samples derived from, for example, at least two patient populations, where one population may be a healthy group of patients and the remaining patient populations may be diseased. Cells may be taken from patients belonging to each patient population and subjected to tests to detect levels of intracellular cholesterol. Such protocol may be repeated (e.g., daily, weekly, biweekly, monthly, every 2 months, every 6 months, and the like) over a length of time (e.g., 1, 2, 3, or more weeks; or 1, 2, 3, 4, 5, 6, or more months; or 1, 2, 3, or 4 more years; etc.). The results of the repeated tests can include a magnitude of the cholesterol defects and/or distress.

The results may then be used to calculate a control level of intracellular cholesterol by comparing the average level of intracellular cholesterol in healthy patients to the average level of intracellular cholesterol in patients with a disease. The control level of intracellular cholesterol may be based on the average level of intracellular cholesterol in patients with a disease. Consequently, when a patient's level of intracellular cholesterol in the test sample may be greater than the control, the patient may have an increased risk of developing Alzheimer's disease. Similarly, when a patient's level of intracellular cholesterol in the test sample may be less than the control, the patient may have a decreased risk of developing Alzheimer's disease. When a patient's level of intracellular cholesterol in the test sample may be substantially equal to the control level, the patient may have to be continuously monitored since cholesterol parameters may be dynamic.

In some embodiments, the test used to determine the control level may be the same as the test used for the patient's test sample. In some embodiments, the filipin test may be used to determine the control level of intracellular cholesterol.

Some embodiments disclosed herein provide for methods for preventing or delaying the onset of Alzheimer's disease, or reducing the symptoms of Alzheimer's disease in a patient in need thereof are also disclosed. The disclosed methods may delay disease onset and/or reduce the symptoms of Alzheimer's disease sufficiently to improve a patient's quality of life. Such methods may include obtaining a test sample from the patient, determining a level of intracellular cholesterol in the test sample, comparing the level of intracellular cholesterol in the test sample with a control level of intracellular cholesterol, identifying at least one cholesterol defect of the patient, and administering at least one therapeutic agent which targets the at least one cholesterol defect.

In some embodiments, the operations of obtaining a test sample from the patient, determining a level of intracellular cholesterol in the test sample, and comparing the level of intracellular cholesterol in the test sample with a control level of intracellular cholesterol may be carried out as described above.

In some embodiments, identifying at least one cholesterol defect may include following up an initial positive result from a test, for example such as a filipin test with further testing for abnormalities in other compounds including, but not limited to, vitamin D metabolites and oxidized forms of cholesterol. In some variations, these tests may be performed simultaneously using flow cytometry to provide even more information about the specific cholesterol defect. Non-limiting examples of the at least one cholesterol defect may include abnormal amounts of cholesterol, its precursors, and/or metabolites; aberrant subcellular localization of cholesterol, its precursors, and/or metabolites; increased levels of oxidized forms of cholesterol; or abnormalities in vitamin D metabolism, all of which may occur separately or in some combination.

In some embodiments, administering at least one therapeutic agent which targets the at least one cholesterol defect may include administering a therapeutic regime directed for a particular patient. At least one therapeutic agent may be administered according to appropriate methods, e.g. orally, intravenously, subcutaneously, and the like. The therapeutic agent may be any therapeutic agent capable of targeting at least one cholesterol defect and/or distress. In some embodiments, the therapeutic agent is administered in a therapeutically effective amount. The therapeutic agent may be a cholesterol-lowering drug. Some non-limiting examples are statins (including but not limited to simvastatin and atorvastatin) and niacin. The therapeutic agent may be used in combination with other therapeutic agents. Non-limiting examples are supplementation with vitamin D or antioxidants, histone deacetylase inhibitors (such as vorinostat and panobinostat), and inhibiting synthesis of lipoprotein receptors (such as with rapamycin).

### Kits

Some embodiments disclosed herein provide for a kit for assessing a patient's risk of developing Alzheimer's disease. In some embodiments, the kit may include a tissue collecting assembly; an intracellular cholesterol staining reagent; and a composite control sample. In variations, separate tissue collecting assemblies for test and control samples may be included in the kit.

In some variations, a tissue collecting assembly may comprise any known tools capable of collecting a sample of cells from a patient.

In some variations, the intracellular cholesterol staining reagent may include filipin, BCθ, Amplex® red reagent, and/or any other staining reagent known in the art. In some embodiments, the intracellular cholesterol staining reagent may be used to detect intracellular cholesterol distress or defects. The kit may also include any other compounds, such as buffers, to appropriately use the reagent. In some embodiments, the kit may also include an assay kit having an intracellular cholesterol staining reagent.

In some embodiments, the composite control sample may be used for comparison with the patient's test result obtained when using the kit. The composite control sample may be in any appropriate form to allow such a comparison.

In some embodiments, the kit may also include a tool for visualizing a cholesterol stain. Such a tool may have the ability to carry out any known methods for visualizing a cholesterol stain, such as by fluorescence microscopy or flow cytometry.

### DEFINITIONS

A “patient” as used herein may refer to a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Administration of the therapeutic agents disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly.

In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

Some embodiments include the combination of compounds, therapeutic agents, and/or pharmaceutical compositions described herein. In such embodiments, the two or more agents may be administered at the same time or substantially the same time. In other embodiments, the two or more agents are administered sequentially. In some embodiments, the agents are administered through the same route (e.g. orally) and in yet other embodiments, the agents are administered through different routes (e.g. one agent is administered orally while another agent is administered intravenously).

### EXAMPLES

The following Examples are presented for the purposes of illustration and should not be construed as limitations.

### Example 1

Experimental Protocol: Fibroblasts were obtained from patients suffering from AD and age-matched control patients via Coriell Institute for Medical Research. The samples were prepared and analyzed by a filipin test as follows: Cells were grown to approximately 75% confluence in 6-well plates. On the day of analysis, cells were rinsed with cold PBS and 200 μl of 0.05% trypsin was added to each well. Once the cells lifted from the plate, complete media was added to neutralize the trypsin and the cell suspension was collected. After centrifugation at 2000 rpm for 5 m, media was removed from the cell pellet and cells were resuspended in 500 μl FACS buffer (PBS+0.5% BSA+2 mM EDTA). 0.5 μl of filipin stock (50 mg/mL) was added to FACS buffer for a final filipin concentration of 50 ug/mL; cells were incubated in filipin solution for 30 m on ice and protected from light. After incubation, cells were centrifuged at 2000 rpm for five minutes, supernatant was removed, and the cell pellet was resuspended in 1 mL FACS buffer and filtered through a 35 μm cell strainer.

Flow cytometry was used to analyze the filipin stains. The flow cytometry analysis was performed as follows. Cell suspensions from previous step were analyzed on a MACSQuant flow cytometry analyzer for intensity of filipin fluorescence with excitation at 405 nm.

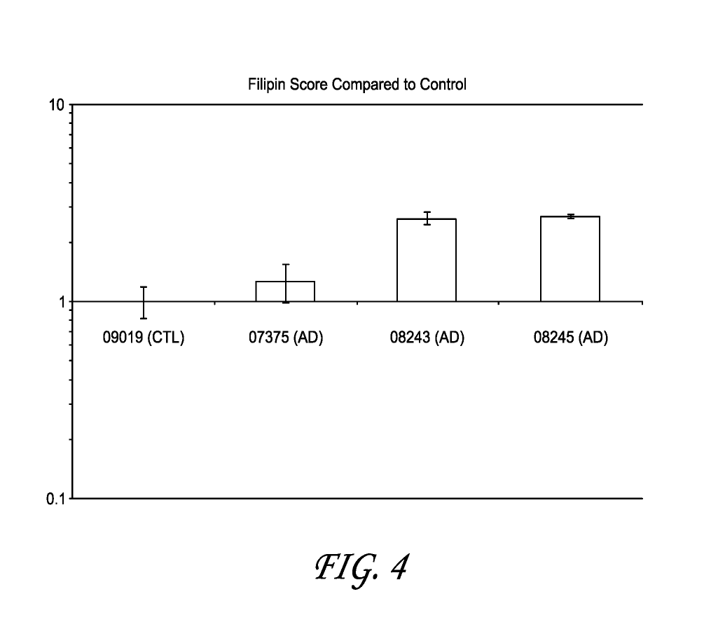

FIG. 4 shows the filipin score of fibroblasts taken from three patients affected with Alzheimer's disease (“08245(AD),” “08243(AD),” “07375(AD)”) and one age-matched cognitively healthy patient (“0919(CTL)”). Filipin score was determined by hand-counting filipin-positive cells. The patient with less DNA damage (07375(AD)) also has a lower filipin score (further description of patients in Table 1). As shown in FIG. 4, 07375(AD) has more filipin staining than control but less than the other two AD patients; this patient also has only 2% random chromosomal loss.

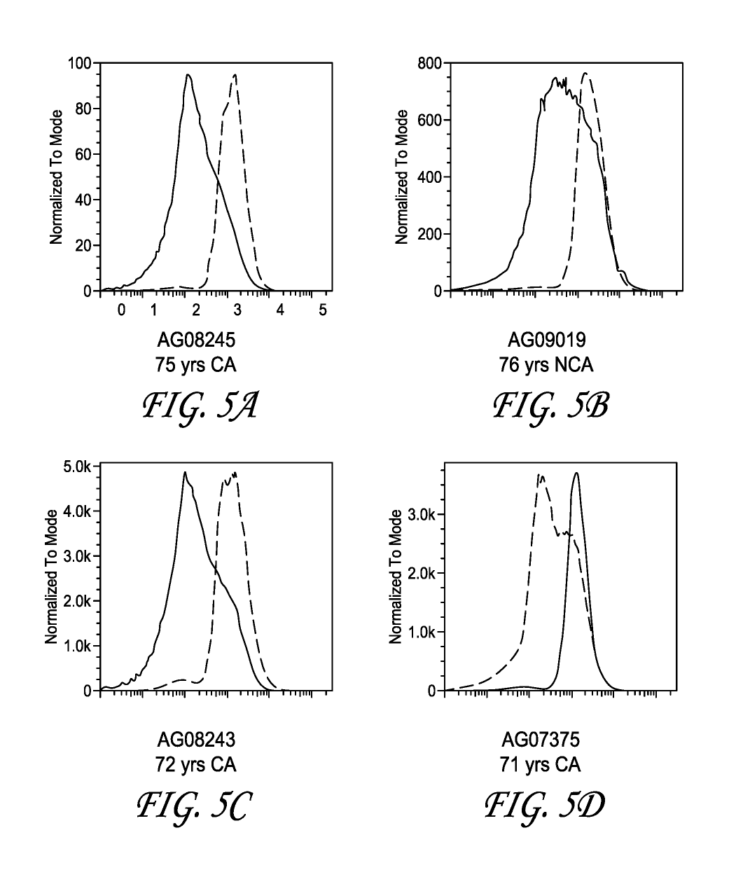

FIG. 5 depicts a fluorescence-activated cell sorting (“FACS”) analysis of fibroblasts taken from three patients affected with Alzheimer's disease (“AG08245,” “AG08243,” “AG07375”) and one age-matched cognitively healthy patient (“AG0919”). The leftmost graph in each panel indicates baseline signal, while the rightmost graph shows filipin intensity. The two peaks are clearly separated in the three clinically affected patients, while in the healthy patient, the two peaks largely overlap. This differential response to filipin may allow for separation of Alzheimer's and healthy patients.

The results of the patients' filipin scores are summarized in the Table 1 below. Samples originating from AD patients showed more intense filipin fluorescence than samples originating from age-matched control patients.

TABLE 1
DESCRIPTION OF PATIENTS AND THEIR FILIPIN PERCENTAGES

| | | Clinically | | Family | Mean |
|---|---|---|---|---|---|
| | Age | affected | Sex | history | % filipin (+) |
| AG07375 | 71 | Yes | Male | No | 40 |
| The donor had a 9 year history of progressive | | | | | |
| mental deterioration. A CT scan at age 67 | | | | | |
| showed marked diffuse cortex cerebral | | | | | |
| atrophy. The skin biopsy was taken antemortem | | | | | |
| from the upper left arm. The culture | | | | | |
| was initiated on Feb. 29, 1984 using explants of | | | | | |
| minced skin tissue. The cell morphology is | | | | | |
| fibroblast-like. The karyotype is 46,XY with 6% | | | | | |
| of the cells examined showing 45,X and 2% | | | | | |
| showing random chromosome loss. | | | | | |
| AG08243 | 72 | Yes | Male | No | 83 |
| The donor is a clinically affected Alzheimer's | | | | | |
| disease patient. The skin biopsy was taken | | | | | |
| ante-mortem from the forearm. Culture was | | | | | |
| initiated on Jun. 11, 1985 using explants of minced | | | | | |
| skin. The cell morphology is fibroblastlike. | | | | | |
| Culture was frozen at PDL 10. The culture is a | | | | | |
| mosaic with karyotype: 46,XY/47,XY,+mar; | | | | | |
| 94%/6% with 10% of the cells examined | | | | | |
| showing random chromosome loss/gain. A | | | | | |
| lymphoblast culture from same donor is | | | | | |
| AG08242. The family history is negative for | | | | | |
| other cases of Alzheimer's disease. | | | | | |
| AG09019 | 76 | No | Female | Yes | 31 |
| The donor is a clinically unaffected probable | | | | | |
| escapee. Father and 11 siblings were affected | | | | | |
| with Alzheimer's disease. The skin biopsy was | | | | | |
| taken ante-mortem from the forearm. Culture | | | | | |
| was initiated on Jul. 8, 1986 using explants of | | | | | |
| minced skin tissue. The cell morphology is | | | | | |
| fibroblast-like. Culture was frozen at PDL 6. | | | | | |
| AG08245 | 75 | Yes | Male | No | 84 |
| The donor had a history of dementia with a | | | | | |
| gradual decline in mental function since age 68 | | | | | |
| years. Autopsy of brain revealed neuritic | | | | | |
| plaques, neurons with ‘tangles’ and the | | | | | |
| granulovacuolar change of Simcowicz | | | | | |
| consistent with Alzheimer's disease. Indications | | | | | |
| of Parkinson's disease were also found | | | | | |
| including degeneration of the substantia nigra | | | | | |
| and locus coeruleus and neurons with Lewy | | | | | |
| bodies. The biopsy was taken post-mortem on | | | | | |
| Jun. 9, 1985. The culture was initiated using explants | | | | | |
| of minced skin tissue. The cell morphology is | | | | | |
| fibroblast-like. The karyotype is 46,XY; normal | | | | | |
| diploid male with 6% of the cells examined | | | | | |
| showing random chromosome loss. A | | | | | |
| lymphoblast culture from same donor is | | | | | |
| AG08244. | | | | | |

### Example 2

The following is a non-limiting example of determining a control level of intracellular cholesterol.

Two patient groups can be formed, one having patients suffering from MCI and the second having age-and-sex matched healthy controls to the first group. Each group can be of sufficient size for proper statistical analysis. Each patient group may also have a wide range of ages represented to aid in determining the risk at the average age that MCI occurs and if there is any age related variability that may also be informative. The MCI patients also may be followed for conversion to AD. This data can be used to further refine the test while validating its predictive value.

Samples from each patient may be obtained according to the method described in Example 1. Then, a filipin test for each sample may be carried out according to the method described in Example 1. The test may then be analyzed by flow cytometry as described in Example 1, and a filipin score can then be determined.

The sample collection and subsequent analysis may be repeated for each patient every 6 months over a three year span. Proper statistical analysis may then be performed on the data. Table 2 provides a summary of exemplary results of filipin scores of patients suffering from MCI versus healthy patients. For comparison, patients with AD are also shown.

TABLE 2
FILIPIN SCORES OF HEALTHY PATIENTS, MCI PATIENTS, AND AD
PATIENTS
Filipin Intensity (units)

| Patient | | | | | | | AD |
|---|---|---|---|---|---|---|---|
| ID | Replicate 1 | Replicate 2 | Replicate 3 | Average | Stdev | Presentation | Risk |
| 1 | 9.96E+01 | 1.06E+02 | 1.13E+02 | 1.06E+02 | 6.63582827 | Healthy | NONE |
| 2 | 1.21E+02 | 9.32E+01 | 1.09E+02 | 1.08E+02 | 14.0591795 | Healthy | NONE |
| 3 | 8.69E+01 | 1.16E+02 | 9.63E+01 | 9.98E+01 | 14.9851172 | Healthy | NONE |
| 4 | 1.08E+02 | 8.28E+01 | 1.04E+02 | 9.83E+01 | 13.5725631 | Healthy | NONE |
| 5 | 1.14E+02 | 9.35E+01 | 1.05E+02 | 1.04E+02 | 10.1491377 | Healthy | NONE |
| 6 | 9.98E+01 | 9.30E+01 | 1.12E+02 | 1.02E+02 | 9.83049011 | Healthy | NONE |
| 7 | 1.14E+02 | 1.00E+02 | 1.07E+02 | 1.07E+02 | 7.17276008 | Healthy | NONE |
| 8 | 1.09E+02 | 1.03E+02 | 1.05E+02 | 1.06E+02 | 3.01199531 | Healthy | NONE |
| 9 | 1.08E+02 | 9.76E+01 | 8.17E+01 | 9.59E+01 | 13.3857732 | Healthy | NONE |
| 10 | 1.03E+02 | 9.60E+01 | 8.75E+01 | 9.55E+01 | 7.77519278 | Healthy | NONE |
| 11 | 1.20E+03 | 1.20E+03 | 1.19E+03 | 1.20E+03 | 9.49655156 | AD | HIGH |
| 12 | 1.19E+03 | 1.22E+03 | 1.23E+03 | 1.21E+03 | 18.5050578 | AD | HIGH |
| 13 | 1.19E+03 | 1.20E+03 | 1.21E+03 | 1.20E+03 | 9.52415367 | AD | HIGH |
| 14 | 1.23E+03 | 1.21E+03 | 1.20E+03 | 1.21E+03 | 14.6722401 | AD | HIGH |
| 15 | 1.21E+03 | 1.19E+03 | 1.22E+03 | 1.21E+03 | 15.1264523 | AD | HIGH |
| 16 | 1.20E+03 | 1.19E+03 | 1.19E+03 | 1.19E+03 | 7.33169494 | AD | HIGH |
| 17 | 1.19E+03 | 1.20E+03 | 1.19E+03 | 1.19E+03 | 4.54291668 | AD | HIGH |
| 18 | 1.22E+03 | 1.20E+03 | 1.20E+03 | 1.21E+03 | 7.33505516 | AD | HIGH |
| 19 | 1.20E+03 | 1.20E+03 | 1.18E+03 | 1.19E+03 | 11.5369586 | AD | HIGH |
| 20 | 1.19E+03 | 1.19E+03 | 1.21E+03 | 1.20E+03 | 11.9137032 | AD | HIGH |
| 21 | 9.01E+02 | 8.46E+02 | 9.10E+02 | 8.86E+02 | 34.8204108 | MCI | LOW |
| 22 | 8.77E+02 | 9.14E+02 | 8.97E+02 | 8.96E+02 | 18.4580421 | MCI | LOW |
| 23 | 9.37E+02 | 8.81E+02 | 8.97E+02 | 9.05E+02 | 28.9243873 | MCI | HIGH |
| 24 | 8.89E+02 | 9.16E+02 | 8.79E+02 | 8.95E+02 | 19.252498 | MCI | LOW |
| 25 | 9.00E+02 | 9.17E+02 | 9.18E+02 | 9.12E+02 | 9.87738419 | MCI | HIGH |
| 26 | 8.72E+02 | 8.98E+02 | 8.85E+02 | 8.85E+02 | 13.0748239 | MCI | LOW |
| 27 | 9.08E+02 | 8.74E+02 | 8.89E+02 | 8.91E+02 | 16.9444921 | MCI | LOW |
| 28 | 8.86E+02 | 8.87E+02 | 8.88E+02 | 8.87E+02 | 1.21635667 | MCI | LOW |
| 29 | 8.96E+02 | 8.85E+02 | 8.93E+02 | 8.91E+02 | 5.82703634 | MCI | LOW |
| 30 | 8.83E+02 | 9.06E+02 | 9.30E+02 | 9.06E+02 | 23.4597286 | MCI | HIGH |
| 31 | 9.14E+02 | 8.66E+02 | 9.33E+02 | 9.04E+02 | 34.3154036 | MCI | HIGH |
| 32 | 9.00E+02 | 8.92E+02 | 9.42E+02 | 9.11E+02 | 27.1093561 | MCI | HIGH |
| 33 | 9.04E+02 | 9.25E+02 | 8.83E+02 | 9.04E+02 | 21.12291 | MCI | HIGH |
| 34 | 8.95E+02 | 8.91E+02 | 9.10E+02 | 8.99E+02 | 9.73495264 | MCI | LOW |
| 35 | 9.09E+02 | 9.02E+02 | 9.31E+02 | 9.14E+02 | 15.01593 | MCI | HIGH |
| 36 | 8.96E+02 | 8.69E+02 | 8.93E+02 | 8.86E+02 | 14.6809799 | MCI | LOW |
| 37 | 8.95E+02 | 8.76E+02 | 9.18E+02 | 8.96E+02 | 21.1672445 | MCI | LOW |
| 38 | 9.12E+02 | 8.95E+02 | 9.44E+02 | 9.17E+02 | 24.8250434 | MCI | HIGH |
| 39 | 9.24E+02 | 9.24E+02 | 8.85E+02 | 9.11E+02 | 22.2799223 | MCI | HIGH |
| 40 | 8.74E+02 | 8.99E+02 | 9.26E+02 | 9.00E+02 | 26.0370983 | MCI | LOW |
| 41 | 8.89E+02 | 8.98E+02 | 9.38E+02 | 9.08E+02 | 26.2701682 | MCI | HIGH |
| 42 | 9.24E+02 | 9.18E+02 | 8.97E+02 | 9.13E+02 | 14.2386274 | MCI | HIGH |
| 43 | 9.01E+02 | 8.88E+02 | 9.06E+02 | 8.99E+02 | 9.2764691 | MCI | LOW |
| 44 | 8.94E+02 | 9.17E+02 | 9.20E+02 | 9.10E+02 | 14.4176074 | MCI | HIGH |
| 45 | 9.10E+02 | 9.17E+02 | 8.76E+02 | 9.01E+02 | 22.1471353 | MCI | HIGH |
| 46 | 9.15E+02 | 9.11E+02 | 9.44E+02 | 9.23E+02 | 17.7837062 | MCI | HIGH |
| 47 | 9.12E+02 | 8.93E+02 | 8.99E+02 | 9.01E+02 | 9.84862052 | MCI | HIGH |
| 48 | 8.90E+02 | 9.08E+02 | 9.14E+02 | 9.04E+02 | 12.2407086 | MCI | HIGH |
| 49 | 8.69E+02 | 9.15E+02 | 9.15E+02 | 9.00E+02 | 26.1506108 | MCI | LOW |
| 50 | 8.60E+02 | 9.09E+02 | 8.88E+02 | 8.86E+02 | 24.799636 | MCI | LOW |

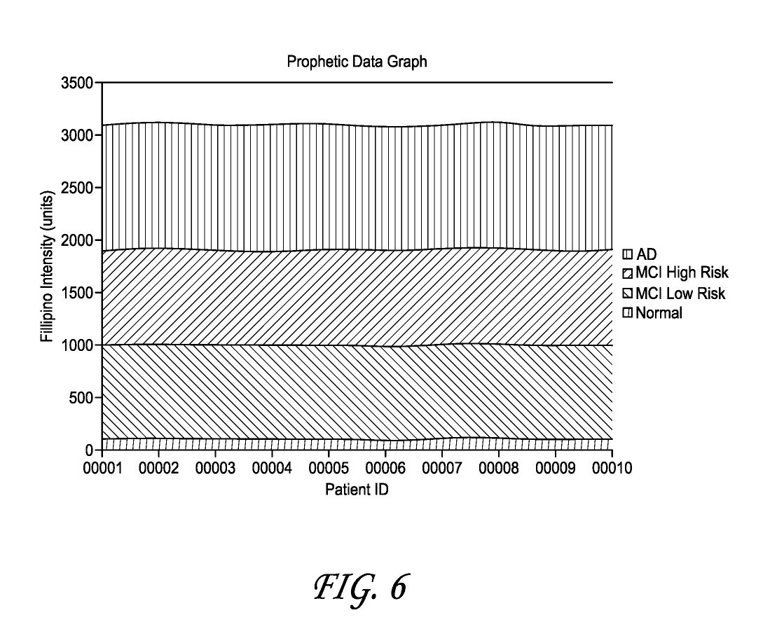

The data can then be graphed to help identify a control level of intracellular cholesterol. For example, FIG. 6, which graphically shows the data summarized in Table 2, shows that Alzheimer's Disease patients can have the highest filipin score, while healthy patients can have the lowest filipin score. Patients presenting with Mild Cognitive Impairment may then be sorted into low and high risk of developing Alzheimer's Disease based on their filipin score.

In some embodiments, peripheral blood cells can be isolated and filipin levels measured by flow cytometry. This can allows for the cells to be isolated inexpensively and at minimal risk to the patient from simple venipuncture rather than requiring the invasive punch biopsy method for collection of fibroblasts as used and described in the results presented herein. A venipuncture can be performed with a venipuncture assembly including, for example, but not limited to, a plastic hub, a hydrodermic needle, a vacuum tube, a syringe, a butterfly needle, and/or other blood collection or vanipuncture devices known in the art. Accordingly, the ability to isolate peripheral blood cells and measure the filipin levels by flow cytometry can facilitate the commercialization of the testing procedure and techniques described herein for preventing and detecting disease.

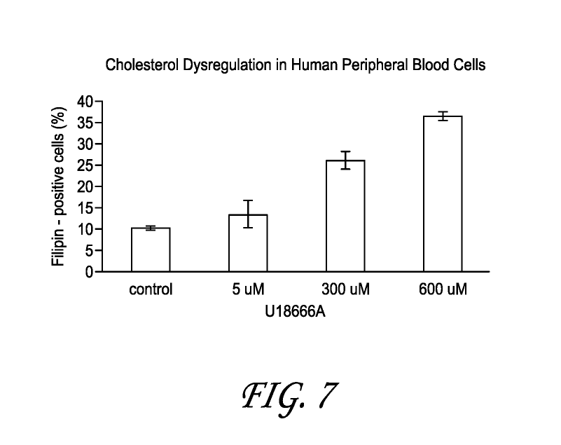

The isolation of peripheral blood cells and measuring of the filipin levels by flow cytometry was tested in two ways. First, testing was conducted to determine whether filipin fluorescence was detectable in peripheral blood cells (PBCs) and whether it displays a range of levels that corresponded to cholesterol dysregulation, as was found with other cell types described in detail herein. Cholesterol dysregulation was induced by treating the cells with Ul 8666A, a compound that inhibits cholesterol synthesis and intracellular trafficking. FIG. 7 shows that a dose-dependent effect of Ul 8666A can be demonstrated on filipin staining in peripheral blood cells, indicating that dysfunction of cholesterol metabolism is capable of occurring in these cells and that the dysfunction of cholesterol metabolism in the peripheral blood cells can be measured by filipin fluorescence.

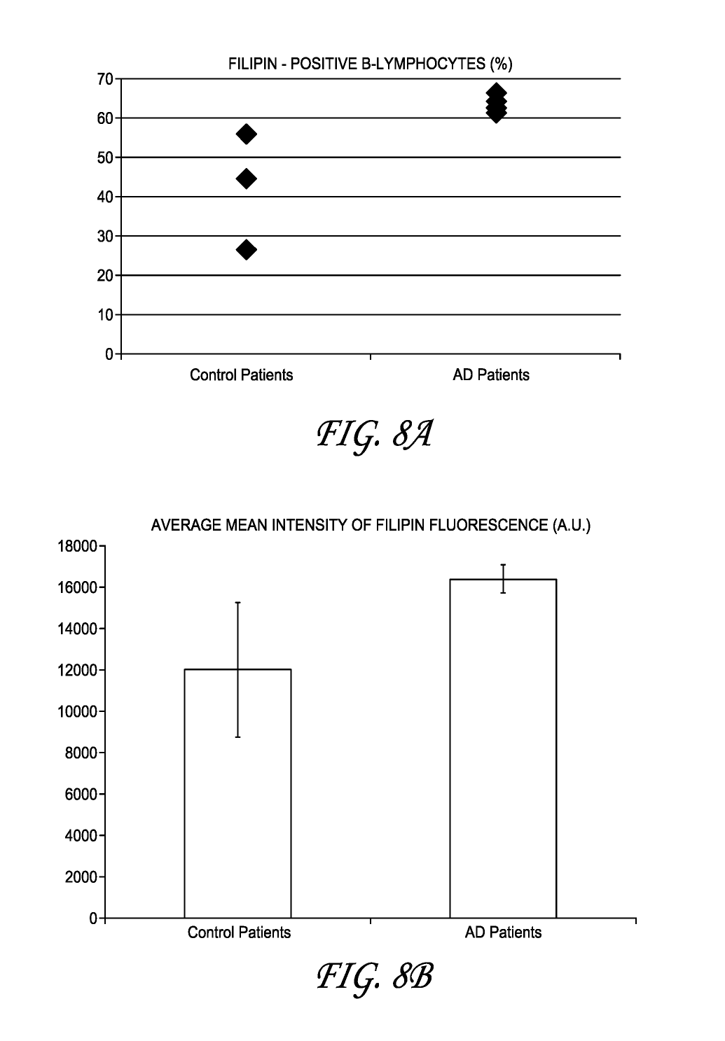

Additionally, a test was conducted which used a subtype of peripheral blood cells, B-lymphocytes, obtained from control and AD patients. These cells were obtained from a cell repository. Using these cells, it was shown that filipin fluorescence alone is capable of differentiating the two groups, control and AD patients. FIGS. 8A-B show the Filipin-positive B-Lymphocytes and the average mean intensity of filipin fluorescence. In FIG. 8A a cutoff was set based on the control cells and it was shown that the AD cells had a higher percentage of filipin-positive cells. FIG. 8B shows the mean fluorescence intensity between the two groups and it was demonstrated that the AD cells had greater overall fluorescence. The results in FIG. 8A-B show that in AD patients, more cells are stained with filipin and that stain is brighter.

In addition, B-lymphocytes were tested from a small population of AD individuals and appropriate controls, and the results shown in FIG. 8A-B demonstrate that the levels of filipin are significantly higher in the AD population.

Therefore, the techniques described herein for detection and/or prevention of disease can be viable in peripheral blood cells isolated from routine venipuncture blood collections. The techniques and/or methods described herein can allow filipin levels to be differentiated between control and AD patients and predict the risk of conversion from MCI to AD. This can be a test and/or technique for clinical use for the identification and clinical management of individuals at high risk of developing AD, as well as a diagnostic tool for the disease.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Various numerical examples, tables, graphs, and data are presented herein. These numerical examples, tables, graphs, and data are intended to illustrate certain example embodiments and not intended to limit the scope of the disclosed methods and kits.

The various features, kits, and methods described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence or order, and the blocks or operations relating thereto can be performed in other sequences or orders that are appropriate. For example, described blocks or operations may be performed in an order other than that specifically disclosed, or multiple blocks or operations may be combined in a single block or operation. The example blocks or operations may be performed in serial, in parallel, or in some other manner. Blocks or operations may be added to, removed from, or rearranged compared to the disclosed example embodiments. The example kits and components described herein may be configured differently than described. For example, elements may be added to, removed from, or rearranged compared to the disclosed example embodiments.

With respect to the use of substantially any plural and/or singular terms herein, those having skill in the art can translate from the plural to the singular and/or from the singular to volume of wastewater can be received in the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

What is claimed is:

1. A method for determining a patient's risk of developing Alzheimer's disease, comprising:
obtaining a test sample from the patient;
combining the test sample with an intracellular cholesterol staining reagent to form a labeled cholesterol complex;
estimating a concentration of intracellular cholesterol in the test sample by quantifying the amount of labeled cholesterol complex in the test sample; and
comparing the estimated concentration of intracellular cholesterol in the test sample with a concentration of intracellular cholesterol in a control sample, wherein a greater concentration of intracellular cholesterol in the test sample compared to the control sample indicates an increased risk of developing Alzheimer's disease.

2. The method of claim 1, wherein the patient suffers from mild cognitive impairment.

3. The method of claim 1, wherein the intracellular cholesterol staining reagent is selected from filipin, BCθ and Amplex® red reagent.

4. The method of claim 3, wherein the staining reagent is filipin.

5. The method of claim 4, wherein flow cytometry is used to quantify the amount of filipin-labeled cholesterol complex in the test sample.

6. The method of claim 5, wherein the test sample comprises peripheral blood cells.

7. A method for preventing or delaying the onset of Alzheimer's disease, or reducing the symptoms of Alzheimer's disease in a patient in need thereof, comprising:
obtaining a test sample from the patient;
determining a level of intracellular cholesterol in the test sample; and
comparing the level of intracellular cholesterol in the test sample with a control level of intracellular cholesterol,
identifying at least one cholesterol defect or distress of the patient; and
administering at least one therapeutic agent which targets the at least one cholesterol defect or distress.

8. The method of claim 7, wherein the patient suffers from mild cognitive impairment.

9. The method of claim 7, wherein a filipin test is used to determine the level of intracellular cholesterol.

10. The method of claim 7, wherein said at least one therapeutic agent is a cholesterol-lowering drug.

11. The method of claim 7, wherein the test sample comprises peripheral blood cells.

12. A kit for assessing a patient's risk of developing Alzheimer's disease, the kit comprising: a tissue collecting assembly; an intracellular cholesterol staining reagent; and a composite control slide.

13. The kit of claim 12, further comprising a tool for visualizing a cholesterol stain.

14. The kit of claim 12, wherein the tissue collecting assembly comprises a venipuncture assembly to collect peripheral blood cells as a test sample.
